(19) United States
(12) Patent Application Publication
(10) Pub. No.: US 2006/0142719 A1
(43) Pub. Date: Jun. 29, 2006

(54) Evaporative disposable garment

(75) Inventors: Robert E. Vogt, Neenah, WI (US); Michael J. Garvey, Appleton, WI (US); Paul T. Van Gompel, Hortonville, WI (US); Thomas H. Roessler, Appleton, WI (US); Rebecca D. Hayes, Appleton, WI (US)

Correspondence Address: SENNIGER POWERS, ONE METROPOLITAN SQUARE, 16TH FLOOR, ST LOUIS, MO, 63102, US

(73) Assignee: Kimberly-Clark Worldwide, Inc., Neenah, WI (US)

(21) Appl. No.: 11/023,710

(22) Filed: Dec. 28, 2004

(51) Int. Cl. A61F 13/15

(52) U.S. Cl. 604/378

(57) ABSTRACT

A disposable garment having a liquid permeable liner, a liquid impermeable/vapor permeable outer cover in opposed relationship with the liner and an absorbent body disposed between the liner and the outer cover. The outer cover has a water vapor transmission rate (WVTR) as determined by a Water Vapor Transmission Rate Test of at least about 6,000 g/m²/24 hours. The disposable garment has an evaporation rate as determined by an Evaporation Rate Test of at least 0.15 g/hr/in². A disposable garment is also disclosed having an absorbent body with an evaporation rate as determined by the Evaporation Rate Test of at least 0.30 g/hr/in², a saturation capacity ratio as determined by a Saturation Capacity Test of at least about 17 grams/gram, and a saturation capacity as determined by the Saturation Capacity Test of at least about 50 grams.

### BACKGROUND OF THE INVENTION

The present invention relates generally to disposable garments such as those used as personal care products, and more particularly to such a disposable garment configured to facilitate evaporation of liquid body waste from the garment following liquid insult thereof.

Absorbent garments find widespread use as personal care products such as diapers, children's toilet training pants, adult incontinence garments and feminine care products. These garments take-in and retain body waste and are typically disposable in the sense that they are intended to be discarded after a limited period of use; i.e., the garments are not intended to be laundered or otherwise restored for reuse. Conventional disposable absorbent garments comprise an absorbent body disposed between a liner adapted for contiguous relationship with the wearer's body and an outer cover for inhibiting liquid body waste taken-in by the absorbent body from leaking out of the article. The liner of the absorbent article is typically liquid permeable to permit liquid body waste to pass therethrough for absorption by the absorbent body.

Because most, if not all, of the liquid body waste taken into an absorbent garment is retained in the absorbent body, the weight and bulk of the garment increases upon repeated insults and restricts movement of the child. Also, because of the increased weight, the garment tends to sag and droop on the wearer's body causing gaps to form between the garment and the wearer's body which often lead to premature leakage and poor fit. Liquid body waste taken in and retained by the garment also contributes to the overall relative humidity near the wearer's skin. High relative humidity in the environment contacting the wearer's skin is a common cause of diaper dermatitis, commonly known as diaper rash.

Prior attempts at reducing the incidence of diaper rash by controlling relative humidity have generally involved adding materials to the garment or by increasing the breathability of the outer cover. However, most of the liquid was still taken into and retained by the absorbent body, thereby still resulting in substantially increasing the weight and bulk of the garment.

There is a need, therefore, for a disposable garment capable of evaporating liquid body waste from the garment following an insult thereof to both reduce humidity near the wearer's skin and reduce the weight increase of the garment due to repeated insults.

### SUMMARY OF THE INVENTION

In one embodiment, a disposable garment of the present invention has a liquid permeable liner having a bodyfacing surface for facing a wearer of the garment and a liquid impermeable and vapor permeable outer cover in opposed relationship with the liner. The outer cover has a water vapor transmission rate (WVTR) as determined by a Water Vapor Transmission Rate Test of at least about 6,000 g/m²/24 hours. An absorbent body is disposed between the liner and the outer cover. The disposable garment has an evaporation rate as determined by an Evaporation Rate Test of at least 0.15 g/hr/in².

In another embodiment of the invention, the disposable absorbent garment has a liquid permeable liner having a bodyfacing surface for facing a wearer of the garment and a liquid impermeable and vapor permeable outer cover in opposed relationship with the liner. The outer cover has a water vapor transmission rate (WVTR) as determined by a Water Vapor Transmission Rate Test of at least about 6,000 g/m²/24 hours. An absorbent body disposed between the liner and the outer cover has an evaporation rate as determined by an Evaporation Rate Test of at least 0.30 g/hr/in².

In yet another embodiment of the present invention, the disposable garment has a liquid permeable liner having a bodyfacing surface for facing a wearer of the garment and a liquid impermeable and vapor permeable outer cover in opposed relationship with the liner. An absorbent body is disposed between the liner and the outer cover, the absorbent body having an evaporation rate as determined by an Evaporation Rate Test of at least 0.30 g/hr/in², and a saturation capacity ratio as determined by a Saturation Capacity Test of at least about 17 grams/gram. The disposable garment has an evaporation rate as determined by an Evaporation Rate Test of at least 0.15 g/hr/in².

A disposable garment for personal wear according to another embodiment comprises a liquid permeable liner having a bodyfacing surface for facing a wearer of the garment, a liquid impermeable and vapor permeable outer cover in opposed relationship with the liner, and an absorbent body disposed between the liner and the outer cover. The absorbent body has an evaporation rate as determined by an Evaporation Rate Test of at least 0.30 g/hr/in²., and a saturation capacity as determined by a Saturation Capacity Test of at least about 50 grams. The disposable garment has an evaporation rate as determined by the Evaporation Rate Test of at least 0.15 g/hr/in².

Other features of the invention will be in part apparent and in part pointed out hereinafter

### BRIEF DESCRIPTION OF THE DRAWINGS

FIG. 1 is a top plan view of a disposable garment of the present invention in a stretched and laid flat condition with the surface of the garment which contacts the skin of the wearer facing the viewer;

FIG. 2 is a cross-section taken along the plane including lines 2-2 in FIG. 1;

FIG. 3 is a view similar to FIG. 2 but showing a cross-section of a disposable garment of a second embodiment of the invention; and

FIG. 4 is a view similar to FIG. 2 but showing a cross-section of an disposable garment of a third embodiment of the invention.

Corresponding reference characters indicate corresponding parts throughout the several views of the drawings.

### DETAILED DESCRIPTION OF THE INVENTION

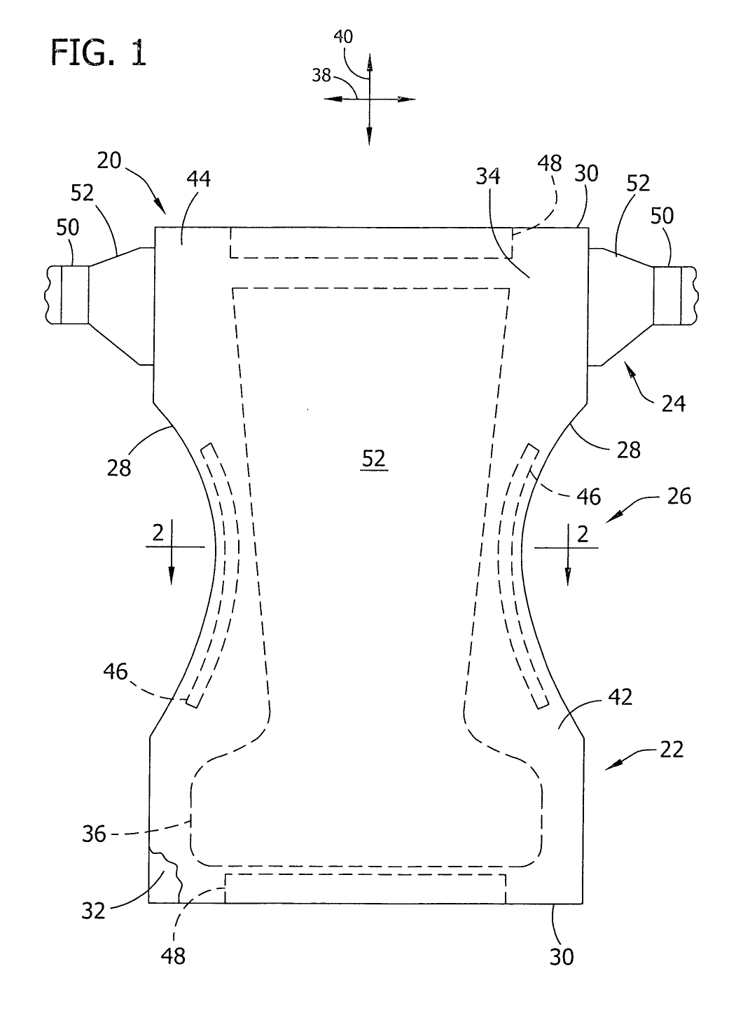

Referring now to the drawings and in particular to FIG. 1, one example of a disposable garment constructed in accordance with the present invention is illustrated in the form of a diaper, which is indicated in its entirety by the reference numeral 20. As used herein, disposable garments are intended to be discarded after a limited period of use instead of being laundered or otherwise restored for reuse. As will be more fully explained below, the diaper 20 is configured to facilitate evaporation of the liquid body waste from the diaper following insult to reduce relative humidity in the environment surrounding the wearer's skin and to maintain the diaper relatively lightweight and flexible following insult. It is therefore understood that the features of the present invention are equally adaptable to other types of disposable garments such as adult incontinence garments, toilet training pants, feminine care products and the like.

The disposable diaper 20 is shown in FIG. 1 in an unfolded and laid flat condition to illustrate a longitudinal axis 40 and lateral axis 38 of the diaper. The diaper 20 generally has a front (e.g., anterior) region 22, a rear (posterior) region 24, and a center region, otherwise referred to herein as a crotch region 26, extending longitudinally between and interconnecting the front and back waist regions 22, 24. The diaper 20 shown in FIG. 1 is generally I-shaped, and more particularly hourglass shaped, and has contoured, laterally opposite side edges 28 and longitudinally opposite front and rear waist ends 30. It is understood, however, that the diaper 20 may have other shapes, such as a rectangular shape or a T-shape without departing from the scope of the present invention. The side edges 28 of the diaper 20 extend longitudinally from the front region 22 through the crotch region 26 to the back region 24 for forming transversely spaced leg openings of the diaper when worn.

The front region 22 generally includes the portions of the diaper 20 that extend over the wearer's lower abdominal region and the back region 24 generally includes the portions of the diaper that extend over the wearer's lower back region. The crotch region 26 includes the portion extending longitudinally through the wearer's crotch from the front region 22 to the back region 24 and laterally between the wearer's legs. As worn on the wearer's body, the diaper 20 further defines a central waist opening of the diaper as well as the leg openings.

The diaper 20 comprises a substantially liquid impermeable outer cover 32, a liquid permeable bodyside liner 34 positioned in facing relation with the outer cover 32, and an absorbent body 36 disposed between the outer cover and the bodyside liner. Marginal portions of the diaper 20, such as marginal sections of the outer cover 32, may extend beyond the terminal edges of the absorbent body 36. In the illustrated embodiment, for example, the outer cover 32 extends outwardly beyond the terminal marginal edges of the absorbent body 36 to form side margins 42 and end margins 44 of the diaper 20. The bodyside liner 34 has a bodyfacing surface and is generally coextensive with the outer cover 32, but may optionally cover an area that is larger or smaller than the area of the outer cover 32, as desired.

To provide improved fit and to help reduce leakage of body exudates from the diaper 20, the side margins 42 and end margins 44 of the diaper may be elasticized with suitable elastic members, such as leg elastic members 46 and waist elastic members 48. For example, the leg elastic members 46 may include single or multiple strands of elastic or elastomeric composites which are constructed to operably gather and shirr the side margins 42 of the diaper 20 to provide elasticized leg bands which can closely fit around the legs of the wearer to reduce leakage and provide improved comfort and appearance. Similarly, the waist elastic members 48 can be employed to elasticize the end margins 44 of the diaper 20 to provide elasticized waistbands. The waist elastics 48 are configured to operably gather and shirr the waistband sections to provide a resilient, comfortably close fit around the waist of the wearer.

The elastic members 46 and 48 are secured to the diaper 20 in an elastically contractible condition so that in a normal under strain configuration, the elastic members effectively contract against the diaper 20. For example, the elastic members 46 and 48 may be elongated and secured to the diaper 20 while the diaper is in an uncontracted condition. In FIG. 1, the elastic members 46 and 48 are shown in their uncontracted, stretched condition for illustrative purposes. The diaper 20 may also include a pair of elasticized, longitudinally extending containment flaps (not shown), which are well know in the art and are typically configured to serve as an additional barrier to the lateral flow of body exudates.

Alternatively, the diaper 20 may include a pair of separate, elasticized and gathered leg gussets (not shown) or combination leg gussets/containment flaps (not shown) which are attached to the diaper along the side margins 42 in at least the crotch region 26 of the diaper 20 to provide elasticized leg cuffs. Such gussets or combination gussets/containment flaps may be configured to extend beyond and bridge across the respective concave portion of the side margins 42.

Fastener tabs 52 (FIG. 1) are secured to the diaper 20 generally at the back region 24 thereof with the tabs extending laterally out from the opposite side edges 28 of the diaper. The fastener tabs 52 may be attached to the outer cover 32, to the bodyside liner 34, between the outer cover and liner, or to other components of the diaper 20. The tabs 52 may also be elastic or otherwise rendered elastomeric. For example, the fastener tabs 52 may be an elastomeric material such as a neck-bonded laminate (NBL) or stretch-bonded laminate (SBL) material.

Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the names of Taylor et al., the disclosures of which are hereby incorporated by reference. Examples of articles that include selectively configured fastener tabs are described in U.S. Pat. No. 5,496,298 issued Mar. 5, 1996 to Kuepper et al.; U.S. Pat. No. 5,540,796 to Fries; and U.S. Pat. No. 5,595,618 to Fries; the disclosures of which are also incorporated herein by reference. Alternatively, the fastener tabs 52 may be formed integrally with a selected diaper component. For example, the tabs 52 may be formed integrally with the outer cover 32, or with the bodyside liner 34.

Fastening components 50, such as hook and loop fasteners (only one set of fasteners are shown in FIG. 1 as being secured to the fastener tabs 52 in the diaper 20 of FIG. 1, the mating fastening components not being shown but being disposed on the outer facing surface of the outer cover 32 at the front region 22 of the diaper), are employed to secure the diaper 20 on the body of a child or other wearer. Alternatively, other fastening components (not shown), such as buttons, pins, snaps, adhesive tape fasteners, cohesives, mushroom-and-loop fasteners, or the like, may be employed. Desirably, the interconnection of the fastening components 50 is selectively releasable and re-attachable. In the illustrated embodiment, the hook fasteners 50 are secured to and extend laterally out from the respective fastener tabs 52 at the back region 24 of the diaper 20. However, it is understood that the fastener tabs 52 may be formed of a hook material and thus comprise the hook fasteners 50 without departing from the scope of this invention. The loop fastener (not shown) is suitably a panel of loop material secured to the outer cover 32 at the front region 22 of the diaper 20 to provide a “fasten anywhere” mechanical fastening system for improved fastening of the hook fasteners 50 with the loop fastener.

The various components of the diaper 20 are assembled together using various suitable attachment techniques, such as adhesive, ultrasonic bonds, pressure bonds, thermal bonds, or combinations thereof. In the illustrated embodiment, for example, the bodyside liner 34 and the outer cover 32 are suitably assembled to each other and to the absorbent body 36 with adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Similarly, other diaper components, such as the elastic members 46 and 48 and the fasteners 50, may be assembled into the diaper 20 by employing the above-identified attachment techniques.

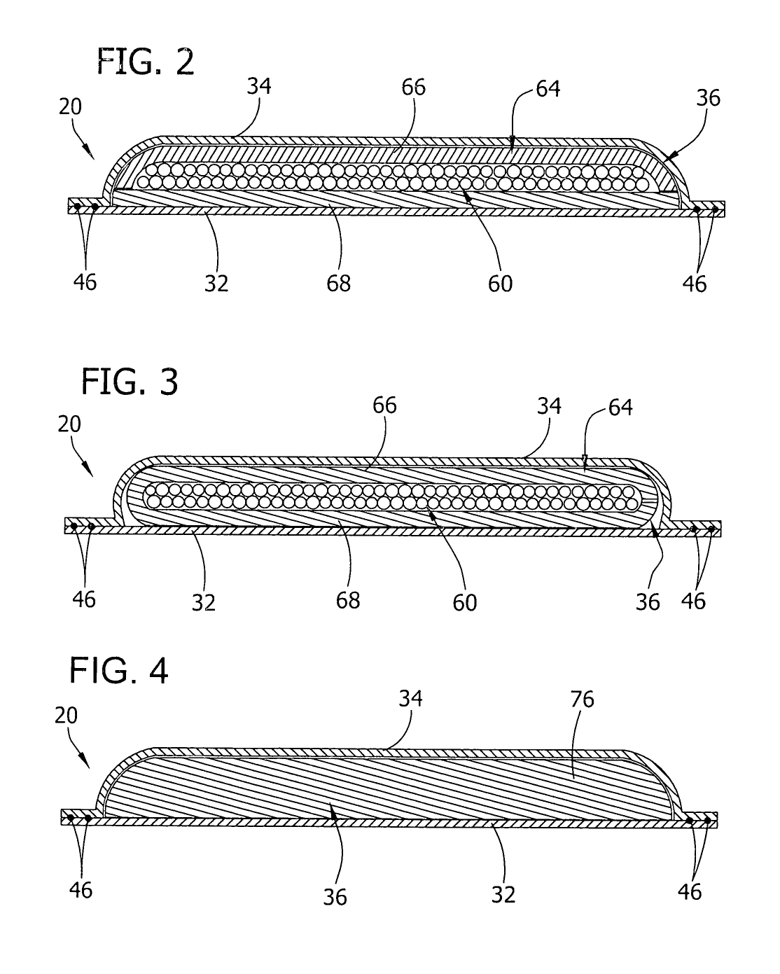

The bodyside liner 34, as representatively illustrated in FIGS. 1 and 2, suitably presents a bodyfacing surface of the diaper 20 which is compliant, soft feeling, and non-irritating to the wearer's skin. Further, the bodyside liner 34 may be less hydrophilic than the absorbent body 36, to present a relatively dry surface to the wearer, and may be sufficiently porous to be liquid permeable, permitting liquid to readily penetrate through its thickness. A suitable bodyside liner 34 may be manufactured from a wide selection of web materials, such as porous foams, reticulated foams, apertured plastic films, natural fibers (i.e., wood or cotton fibers), synthetic fibers (i.e., polyester or polypropylene fibers), or a combination of natural and synthetic fibers. The bodyside liner 34 is suitably employed to help isolate the wearer's skin from liquids held in the absorbent body 36.

Various woven and nonwoven fabrics can be used for the bodyside liner 34. For example, the bodyside liner 34 may be composed of a meltblown or spunbonded web of polyolefin fibers. The bodyside liner 34 may also be a bonded-carded web composed of natural and/or synthetic fibers. The bodyside liner 34 may be composed of a substantially hydrophobic material, and the hydrophobic material may, optionally, be treated with a surfactant, a wetting agent, or otherwise processed to impart a desired level of wettability and hydrophilicity. The bodyside liner 34 may be an elastic material such that the liner is elastically stretchable in the lateral direction 38 and/or the longitudinal direction 40. The term “stretchable” as used herein may include materials that are extensible and materials that are elastic. Suitable elastically stretchable materials are disclosed in U.S. patent application Ser. No. 10/879,323 filed Jun. 29, 2004 and titled “DISPOSABLE GARMENT WITH STRETCHABLE ABSORBENT ASSEMBLY”, the disclosure of which is hereby incorporated by reference.

As one example of a suitable bodyside liner 34 construction, the liner may comprise a nonwoven, spunbond, polypropylene fabric composed of about 2.8-3.2 denier fibers formed into a web having a basis weight of about 20 grams per square meter and a density of about 0.13 gram per cubic centimeter. The fabric may be surface treated with about 0.3 weight percent of a surfactant mixture, which contains a mixture of AHCOVEL Base N-62 and GLUCOPOAN 220UP surfactant in a 3:1 ratio based on a total weight of the surfactant mixture. The AHCOVEL Base N-62 is purchased from Hodgson Textile Chemicals Inc., (Mount Holly, N.C.) and includes a blend of hydrogenated ethoxylated castor oil and sorbitan monooleate in a 55:45 weight ratio. The GLUCOPAN 220UP is purchased from Henkel Corporation and includes alkyl polyglycoside. The surfactant may be applied by any conventional means, such as spraying, printing, brush coating, or the like. The surfactant may be applied to the entire bodyside liner 34, or may be selectively applied to particular sections of the bodyside liner 34, such as the medial section along the longitudinal centerline of the diaper, to provide greater wettability of such sections.

The bodyside liner 34 may further include a lotion or treatment applied thereto that is configured to be transferred to the wearer's skin. Suitable compositions for application to the bodyside liner 34 are described in U.S. Pat. No. 6,149,934 that issued to Krzysik et al. on Nov. 21, 2000 and U.S. patent application Ser. No. 10/660,319 filed Sep. 11, 2003, the disclosures of which are hereby incorporated by reference.

The outer cover 32 of the diaper 20 is suitably constructed to be substantially liquid impermeable but vapor permeable (e.g., “breathable”). Vapor permeability, as used herein in reference to the outer cover 32, refers to the water vapor transmission rate (WVTR) of the outer cover as determined by the following procedure.

Water Vapor Transmission Rate (WVTR) Test

A suitable technique for determining the WVTR (water vapor transmission rate) value of a film or laminate material (e.g., the outer cover 32) is the test procedure standardized by INDA (Association of the Nonwoven Fabrics Industry), number IST-70.4-99, entitled “STANDARD TEST METHOD FOR WATER VAPOR TRANSMISSION RATE THROUGH NONWOVEN AND PLASTIC FILM USING A GUARD FILM AND VAPOR PRESSURE SENSOR” which is incorporated by reference herein. The INDA procedure provides for the determination of WVTR, the permeance of the film to water vapor and, for homogeneous materials, water vapor permeability coefficient.

The INDA test method is well known and will not be set forth in detail herein. However, the test procedure is summarized as follows. A dry chamber is separated from a wet chamber of known temperature and humidity by a permanent guard film and the sample material to be tested. The purpose of the guard film is to define a definite air gap and to quiet or still the air in the air gap while the air gap is characterized. The dry chamber, guard film, and the wet chamber make up a diffusion cell in which the test film is sealed. The sample holder is known as the Permatran-W Model 100K manufactured by Mocon/Modern Controls, Inc., Minneapolis, Minn. A first test is made of the WVTR of the guard film and the air gap between an evaporator assembly that generates 100% relative humidity. Water vapor diffuses through the air gap and the guard film and then mixes with a dry gas flow which is proportional to water vapor concentration. The electrical signal is routed to a computer for processing. The computer calculates the transmission rate of the air gap and the guard film and stores the value for further use.

The transmission rate of the guard film and air gap is stored in the computer as CaIC. The sample material is then sealed in the test cell. Again, water vapor diffuses through the air gap to the guard film and the test material and then mixes with a dry gas flow that sweeps the test material. Also, again, this mixture is carried to the vapor sensor. The computer than calculates the transmission rate of the combination of the air gap, the guard film, and the test material. This information is then used to calculate the transmission rate at which moisture is transmitted through the test material according to the equation:
TR⁻¹_{test material}=TR⁻¹_{test material,guardfilm,airgap}−TR⁻¹_{guardfilm,airgap}
Calculations:
WVTR: The calculation of the WVTR uses the formula:
WVTR=Fpₛₐₜ(T)RH/Apₛₐₜ(T)(1−RH))
where:

F=The flow of water vapor in cc/min.,

pₛₐₜ (T)=The density of water in saturated air at temperature T,

RH=The relative humidity at specified locations in the cell,

A=The cross sectional area of the cell, and,

pₛₐₜ (T)=The saturation vapor pressure of water vapor at temperature T.

In particular, the outer cover 32 is suitably constructed to have a WVTR of at least about 6000 g/m²/24 hours, more suitably at least about 12,000 g/m²/24 hours, and even more suitably at least about 15,000 g/m²/24 hours.

In one particularly suitable embodiment, the outer cover 32 may comprise a microporous film/nonwoven laminate comprising a spunbond nonwoven material layer laminated to a microporous film. For example, the laminate may comprise a 0.6 osy (20.4 gsm) polypropylene spunbond material thermally attached to a microporous film having a density of about 0.88 grams/cubic centimeter (g/cc) and formed from a two-component single resin comprising a linear low density polyethylene and a mettalocene-catalyzed polymer. Such a film may include from about 20 percent to about 75 percent by weight calcium carbonate particulates and the remainder primarily low density polyethylene. The film is then stretched which causes the polyethylene component to stretch while the particulates remain unstretched, thus causing voids to develop around the calcium carbonate particles in the film. Laminates having suitable WVTR values for use as the outer cover 32 of the diaper 20 are disclosed in U.S. patent application Ser. No. 10/335,244 filed Dec. 31, 2002 and entitled “BREATHABLE, EXTENSIBLE FILMS MADE WITH TWO-COMPONENT SINGLE RESINS” and International Publication No. 2004/060982 published Jul. 22, 2004, the disclosures of which are hereby incorporated by reference.

The WVTR of the laminate outer cover 32 may be adjusted based on a number of variables such as: the type of filler (e.g., calcium carbonate or other suitable filler), the coating on the filler (e.g., stearic acid or other larger chain fatty acid), percent weight of filler (typically in the range of 40-70% by weight), type and percent weight of polymer resin (e.g., polyethylene, LDPE, polypropylene, polyolefin copolymers, or other suitable polymers), thickness of film before stretching, temperature of film during stretching, percent draw in the stretching step, direction of stretch (MD, CD, or biaxial), and rate of draw during the stretching step.

Examples of additional suitable breathable materials for use as the outer cover 32 are also described in U.S. Pat. No. 5,855,999 issued Jan. 5, 1999 to McCormack et al. and entitled “BREATHABLE, CLOTH-LIKE FILM/NONWOVEN COMPOSITE”, U.S. Pat. No. 6,045,900 issued Apr. 4, 2000 to Haffner et al. and entitled “BREATHABLE FILLED FILM LAMINATE”, U.S. Pat. No. 6,075,179 issued Jun. 13, 2000 to McCormick et al. and entitled “LOW GAUGE FILMS AND FILM/NONWOVEN LAMINATES”, U.S. Pat. No. 6,600,086 issued Jul. 29, 2003 to Mace et al. and entitled “BREATHABLE DIAPER OUTER COVER WITH FOAM DAMPNESS INHIBITOR”, and U.S. Pat. No. 6,679,869 issued Jan. 20, 2004 to Schlinz et al. and entitled “ABSORBENT ARTICLE HAVING AN ELASTIC OUTER COVER”, the disclosures of which are herein incorporated by reference.

The absorbent body 36 of the diaper 20 is suitably constructed to facilitate evaporation of liquid from the absorbent body, and more suitably to work in conjunction with the breathable outer cover to facilitate evaporation of liquid body waste from the diaper 20. In one embodiment, the absorbent body is also suitably capable of retaining at least some liquid therein following insult.

In the illustrated embodiment of FIGS. 1 and 2, the absorbent body 36 comprises a retention layer, generally indicated 60, surrounded by a liquid permeable wrapsheet, generally indicated 64. In one particularly suitable embodiment, the retention layer 60 comprises superabsorbent material, and more suitably particulate superabsorbent material. As used herein, the term “superabsorbent material” refers to a water-swellable, water-insoluble organic or inorganic material capable, under the most favorable conditions, of absorbing at least about ten times its weight and, more desirably, at least about thirty times its weight in an aqueous solution containing 0.9 weight percent sodium chloride. The superabsorbent material of the retention layer 60 can be selected from natural, synthetic, and modified natural polymers and materials.

The superabsorbent material can comprise inorganic materials, such as silica gels, or organic compounds, such as cross-linked polymers. The term “cross-linked” refers to any means for effectively rendering normally water-soluble materials substantially water insoluble but swellable. Such means can include, for example, physical entanglement, crystalline domains, covalent bonds, ionic complexes and associations, hydrophilic associations, such as hydrogen bonding, and hydrophobic associations or Van der Waals forces. Examples of suitable synthetic superabsorbent material polymers include the acidic or alkali metal and ammonium salts of poly(acrylic acid) and poly (methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), or basic or chloride and hydroxide salts of polyvinyl amine, polyamine polyquarternary ammonium, polyimine, hydrolyzed polyamide, and mixtures and copolymers thereof.

Other suitable superabsorbent material polymers include natural and modified natural polymers, such as hydrolyzed acrylonitrile-grafted starch, acrylic acid grafted starch, methyl cellulose, chitosan, carboxymethyl cellulose, hydroxypropyl cellulose, and the natural gums, such as alginates, xanthan gum, locust bean gum and the like. Mixtures of natural and wholly or partially synthetic absorbent polymers can also be useful in the present invention. Additional suitable superabsorbent materials are disclosed in U.S. Pat. No. 3,901,236 issued Aug. 26, 1975 and processes for preparing synthetic absorbent gelling polymers are disclosed in U.S. Pat. No. 4,076,663 issued Feb. 28, 1978 and U.S. Pat. No. 4,286,082 issued Aug. 25, 1981.

Examples of suitable superabsorbent materials are disclosed in U.S. Pat. Nos. 5,981,689; 6,072,101; 6,087,448; 6,121,409; 6,159,591; 6,194,631; 6,222,091; 6,235,965; 6,342,298; 6,376,072; 6,392,116; 6,509,512; and 6,555,502; U.S. Patent Publications 2001/01312; 2001/07064; 2001/29358; 2001/44612; 2002/07166; 2002/15846; and 2003/14027; and PCT Publications WO 99/25393; WO 99/25745; WO 99/25748; WO 00/56959; WO 00/63295; WO 02/10032; WO 03/18671; and WO 03/37392; the disclosures of which are incorporated by reference to the extent they are consistent herewith.

The superabsorbent material particles used in forming the retention layer 60 can be of any desired configuration, such as spiral or semi-spiral, cubic, rod-like, polyhedral, random, spherical (e.g., beads), needles, flakes, and fibers. Conglomerates of particles of superabsorbent material may also be used in forming the retention layer 60. As an example, in a particularly suitable embodiment the superabsorbent material particles have an average particle size in the range of from about 20 micrometers to about 1 millimeter. “Particle size” as used herein means the weighted average of the smallest dimension of the individual particles.

Still more suitably, the retention layer 60 is free from cellulosic materials (e.g., wood pulp fluff) which have fluid retention properties that are not conducive to evaporation of fluid from the absorbent body 36. Such materials tend to readily absorb and hold fluid and retard the rate at which liquid can be evaporated from the diaper 20. In contrast, superabsorbent material particles take longer to absorb liquid (e.g., compared with fibrous material such as fluff or surge material) and provide more outer surface area from which liquid can be evaporated. It is contemplated that the retention layer 60 may alternatively, or may additionally, comprise foams or other suitable non-cellulosic materials and remain within the scope of this invention.

The liquid permeable wrapsheet 64 surrounding the retention layer 60 defines an inner separation layer 66 disposed between the retention layer and the liner 34, and an outer separation layer 68 disposed between the retention layer and the outer cover 32. In the illustrated embodiment, the outer separation layer 68 lies in direct contact with, and may be adhered to, the outer cover 32. The inner separation layer 66 lies in direct contact with, and may be adhered to, the liner 34. It is contemplated, however, that one or more additional material layers may be disposed between the outer separation layer 68 and the outer cover 32 and/or between the inner separation layer 66 and the liner 34, and remain within the scope of this invention.

In one embodiment, the inner separation layer 66 is less hydrophilic than the retention layer 60. More suitably, the inner separation layer 66 comprises a surge material capable of quickly taking in and temporarily holding a surge of liquid body waste that penetrates the liner 34 upon insult of the diaper 20, and then releasing the liquid body waste for flow to the retention layer 60. The surge material may be composed of a meltblown or spunbonded web of polyolefin fibers. The surge material may also be a bonded-carded-web or an airlaid web composed of natural and synthetic fibers. The bonded-carded-web may, for example, be a powder-bonded-carded web, an infrared bonded carded web, or a through-air-bonded-carded web. The infrared and through-air bonded carded webs can optionally include a mixture of different fibers, and the fiber lengths within a selected fabric web may be within the range of about 2.54-7.62 cm (about 1.0-3.0 inch). The surge material may be composed of a substantially hydrophobic material, and the hydrophobic material may optionally be treated with a surfactant or otherwise processed to impart a desired level of wettability and hydrophilicity.

The surge material may be a fibrous nonwoven web comprising a bonded, uniformly mixed, single layer structure having a basis weight of at least 20 grams per square meter, a void volume between about 40 and 60 cubic centimeters per gram of web at a pressure of 689.10⁻⁵ N (689 dynes) per square centimeter (0.01 psi), a permeability of about 5,000 to about 8,000 darcy, a porosity of about 97.2% to about 98.8% and a surface area per void volume of about 24 to about 49 square centimeters per cubic centimeters. The web fibers may be thermoplastic, and may be heat bonded to one another. In addition, the web structure can have a density within a range of about 0.017-0.025 g/cm³, as determined at a pressure of 689.10⁻⁵ N (689 dynes) per square centimeter (0.01 psi) Other suitable materials for use as the wrapsheet 64 are disclosed in European Patent Application No. EP 0948951 entitled “Absorbent article having improved surge management” and U.S. Pat. Nos. 4,798,603; 5,364,382; 5,429,629; 5,486,166; 5,490,846; 5,522,810; and 5,562,650 the disclosures of which are incorporated by reference herein.

In the illustrated embodiment of FIG. 2, the outer separation layer 68 of the wrapsheet 64 is formed separately from the inner separation layer 66. The inner separation layer 66 extends down over the side edges of the retention layer 60 and is adhered to the outer separation layer 68 to form the wrapsheet surrounding the retention layer. The outer separation layer 68 is suitably constructed of a less hydrophilic material than the retention layer 60, and is more suitably constructed of a surge material, such as same surge material from which the inner separation layer 66 is constructed.

Alternatively, the wrapsheet 64 may comprise a unitary layer wrapped entirely around the retention layer 60 to define the inner and outer separation layers 66, 68 as shown in FIG. 3. It is also contemplated that the wrapsheet 64 may instead extend only partially about the retention layer 60. For example, the wrapsheet 64 may comprise only the inner separation layer 66, or it may comprise only the outer separation layer 68, without departing from the scope of this invention. As shown in the alternative embodiment of FIG. 4, the absorbent body 36 may comprise a retention layer 76 disposed between the liner 34 and outer cover 32 without being surrounded in whole or in part by a wrapsheet.

The surface area of the portion of the absorbent body 36 that faces the outer cover 32 generally defines an evaporative surface area of the diaper 20 over which liquid in the garment may be evaporated therefrom. As an example, where the diaper 20 is sized to fit a typical one-year old child the evaporative surface area of the diaper is approximately 56 square inches (361 square cm). It is understood, however, that the diaper 20 may have more or less evaporative surface area without departing from the scope of this invention.

In use, when the wearer of the diaper 20 insults the diaper (e.g., urinates therein), the liquid insult penetrates the bodyside liner 34 and is quickly taken into the inner separation layer 66 of the absorbent body 36. Some of the liquid passes through the inner separation layer 66 to the retention layer 60, while additional liquid is distributed (e.g., wicks or flows) to other regions of the inner separation layer before passing to the retention layer. Because the superabsorbent material from which the retention layer 60 is constructed does not quickly absorb the liquid (e.g., compared with fibrous surge material), some of the liquid in the retention layer flows down to the outer separation layer 68 while the remainder of the liquid remains within void space in the superabsorbent particles or on the outer surfaces of the superabsorbent particles. Liquid that flows to the outer separation layer 68 contacts the liquid impermeable outer cover 32 and is directed by the outer cover to flow laterally and longitudinally outward to distribute the liquid over the surface area of the absorbent body 36 defined by the outer cover facing portion of the absorbent body (and more particularly of the outer separation layer 68).

While the superabsorbent material absorbs the liquid in the retention layer 60 (and may even draw in liquid from the inner and/or outer separation layers, 66 and 68, respectively), liquid in the outer separation layer, and more particularly at the evaporative surface area of the diaper 20, evaporates out through the outer cover 32 of the diaper. As the outer separation layer 68 dries, unabsorbed liquid in the retention layer 60 flows down to the outer separation layer and is also evaporated. Ambient air may also flow in through the outer cover 32 into contact with the absorbent body 36 and cause liquid in the retention layer 60 to evaporate.

In one embodiment, the diaper 20 has a suitably high evaporation rate so that a substantial volume of liquid is evaporated from the diaper following an insult instead of being retained by the absorbent body. In particularly suitable embodiments, the diaper 20 has an evaporation rate as determined by an Evaporation Rate Test set forth later herein of at least 0.15 g/hr/in² (0.023 g/hr/cm²), more suitably at least about 0.30 g/hr/in² (0.047 g/hr/cm²), and even more suitably at least about 0.40 g/hr/in² (0.062 g/hr/cm²). The absorbent body 36 itself suitably has an evaporation rate as determined by the Evaporation Rate Test of at least 0.30 g/hr/in², more suitably at least 0.35 g/hr/in², and even more suitably at least about 0.40 g/hr/in².

### Evaporation Rate Test

A suitable laboratory test method for determining the evaporation rate of a specimen is described herein. Each specimen to be tested is 2 inch (5.1 cm) by 2 inch (5.1 cm) (e.g., 4 square inches (26 square cm)). Specimens may be formed to the test specimen size or cut from a larger sample. Each specimen to be tested is placed on a flat surface and 6.5 fluid ounces of 0.9 weight percent saline solution is poured from a beaker onto the specimen. The specimen is weighed in its saturated state and then placed on a hot plate such as a Model #SP 18429 hot plate available from Barnstead/Thermplyne, Inc. of Dubuque, Iowa. The hot plate (with the specimen thereon) is maintained at a temperature of 99 degrees F. (37 degrees C.) for a time period of two hours. The specimen is removed from the hot plate and weighed at time intervals of every fifteen minutes to generate a total of eight weight readings over the two hour period for each specimen. Each specimen is tested in an environment having 30% Relative Humidity and an ambient temperature of 73.4 degrees F. (23 degrees C.).

The evaporation rate of the specimen is determined using the following equation:
Evaporation Rate (EV):

$$EV = \frac{{\sum }_{b = 1}^{b = 8} ( \frac{{w}_{b} - {w}_{b + 1}}{Δ t * A} )}{8}$$

$$w - weight of specimen$$

$$b - weight measurement index ( {1}^{st} , {2}^{nd} , etc . )$$

$$Δ t - time interval ( 0.25 hours )$$

$$A - evaporative surface area ( 4 {in}^{2} )$$

In one embodiment, the absorbent body 36 is also constructed to have a suitable saturation capacity ratio as determined by the Saturation Capacity Test described below. In another embodiment, the absorbent body 36 is suitably constructed and sized to have a suitable saturation retention capacity as determined by the Saturation Capacity Test for use in disposable garment such as the diaper 20 described previously herein. The saturation capacity of an absorbent body is a measure of the total amount of liquid that the absorbent body is capable of retaining, and the saturation capacity ratio is a measure of the amount of liquid that an absorbent body is capable of retaining per each gram weight of the absorbent body.

### Saturation Capacity Test

The absorbent body (referred to in this test as the sample) to be tested, having a moisture content of less than about 7 weight percent, is first weighed to determine a dry weight of the sample. The sample is then submerged in an excess quantity of room temperature (about 23 degree C.) synthetic urine as set forth below and remains submerged for 20 minutes. After 20 minutes the sample is removed from the synthetic urine and placed on a TEFLON coated fiberglass screen having 0.25 inch (6.4 mm) openings (commercially available from Taconic Plastics Inc. Petersburg, N.Y.) which, in turn, is placed on a vacuum box and covered with a flexible rubber dam material. A vacuum of 3.5 kilopascals (0.5 pounds per square inch) is drawn in the vacuum box for a period of 5 minutes. The sample is then removed from the vacuum box and weighed against to determine a saturated, or wet weight of the sample. If material, such as superabsorbent material or fiber, is drawn through the fiberglass screen while on the vacuum box, a screen having smaller openings should be used. Alternatively, a piece of the tea bag material described below can be placed between the material and the screen and the final value adjusted for the fluid retained by the material as described below.

The saturation capacity of the sample is determined by subtracting the dry weight of the sample from the wet weight of the sample and is reported in grams of liquid. The saturation capacity ratio of the sample is determined by dividing the saturation capacity by the dry weight of the sample and is stated as grams of fluid retained per gram weight of the sample.

When the material to be tested is particulate superabsorbent material, the test is run as set forth above with the following exceptions. A bag is prepared from heat sealable tea bag material (grade 542, commercially available from the Kimberly-Clark Corporation). A six inch by three inch sample of the tea bag material is folded in half and heat sealed along two edges to form a generally square pouch. 0.2 grams of the superabsorbent material to be tested (in the form of particles having a size within the range of from bout 300 to about 600 microns, and a moisture content of less than about 5 weight percent) is placed in the pouch and the third side is heat sealed. A second pouch is formed without superabsorbent material therein. The above described test is performed on each of the pouches to determine a dry weight of the empty tea bag, a wet weight of the empty tea bag, the dry weight of the sample tea bag (e.g., with the superabsorbent material therein) and the wet weight of the sample tea bag. The saturation capacity of the superabsorbent material is determined by subtracting the wet weight of the empty tea bag and the dry weight of the superabsorbent material from the wet weight of the sample (e.g., filled) tea bag. The saturation capacity is determined as the saturation capacity divided by the dry weight of the superabsorbent material.

The synthetic urine composition referenced herein comprises 0.31 grams monobasic calcium phosphate monohydrate (CaH₄(PO₄)₂H₂O), 0.68 grams monobasic potassium phosphate (KH₂PO₄), 0.48 grams magnesium sulphate heptahydrate (MgSO₄ 7H₂O), 1.33 grams potassium sulphate (K₂ SO₄), 1.24 grams tribasic sodium phosphate dodecahydrate (Na₃ PO₄ 12H₂O), 4.4 grams sodium chloride (NaCl), 3.16 grams potassium chloride (KCl), 8.56 grams of urea (CO(NH₂)₂), 0.1 grams Pluronic 10R8 surfactant (a non-ionic surfactant commercially available from BASF-Wyandotte Corporation) and 1 gram methyl paraben and 1 gram Germall 115 preservative (commercially available from Santell Chemical Company, Chicago, Ill.) per liter using distilled water as the solvent. The components are added to 900 milliliters of distilled water in the order given and each dissolved before the next component is added. The solution is finally diluted to one liter.

In one suitable embodiment, the absorbent body 36 has a saturation capacity ratio as determined by the Saturation Capacity Test set forth above of at least about 15 grams/gram, and more suitably at least about 17 grams/gram, to retain some liquid body waste therein for inhibiting flowback of the liquid body waste back through the liner 34 into contact with the wearer's skin. However, it is contemplated that the absorbent body 36 may have a lower saturation capacity ratio, or may be incapable of retaining liquid, and remain within the scope of this invention. In another embodiment, the absorbent body 36 suitably has a saturation capacity ratio as determined by the Saturation Capacity Test in the range of about 15 grams/gram to about 30 grams/gram, and more suitably about 17 grams/gram to about 30 grams/gram.

The absorbent body 36 according to another suitable embodiment has a saturation capacity as determined by the Saturation Capacity Test of at least 50 grams, and more suitably in the range of about 50 grams to about 500 grams, and even more suitably in the range of about 200 grams to about 500 grams. In another embodiment, the saturation capacity of the absorbent body may be decreased based on the expected evaporation of liquid from the garment in which the absorbent body is incorporated. For example, the saturation capacity of the absorbent body 36 may be in the range of about 50 grams to about 450 grams, more suitably in the range of about 50 grams to about 350 grams, and even more suitably in the range of about 50 grams to about 250 grams.

For comparison purposes, existing HUGGIES Size 3 diapers, available from Kimberly-Clark Worldwide, Inc. of Neenah, Wis., have a theoretical absorbent body saturation capacity of approximately 404 grams of 0.9 molar saline. The theoretical saturation capacity is determined by assuming a saturatation capacity ratio of 8 grams/gram for cellulosic fluff and 30 grams/gram for superabsorbent material present in the absorbent body of the existing diaper. About 10 grams of superabsorbent material is present in the absorbent body, along with 13 grams of cellulosic fluff, providing a theoretical saturation capacity of about 404 grams. It is understood, however, that the above diaper does not have a suitable evaporation rate in accordance with the present invention.

### Experiment 1

A first test was conducted to determine the evaporation rates of nine different absorbent body constructions. Specimen 1 was a control specimen representative of a prior art absorbent body construction comprising 43% by weight HYSORB 8800 superabsorbent material available from BASF Corporation of Charlotte, N.C. and 57% by weight Bowater CR-1654 wood pulp available from Bowater Incorporated of Greenville, S.C. This sample represents the absorbent body construction of absorbent bodies found in HUGGIES SUPREME diapers available from Kimberly-Clark Worldwide, Inc. of Neenah, Wis.

Specimen 2 was a 3.5 ounces per square yard (osy) (119 grams per square meter (gsm)) Through Air Bonded Carded Web (TABCW) material commonly used as surge layer material on conventional diapers such as diapers manufactured by Kimberly-Clark Worldwide, Inc. of Neenah, Wis. under aforementioned U.S. Pat. No. 5,562,650.

Specimen 3 was a layer of HYSORB 8800 superabsorbent material available from BASF Corporation of Charlotte, N.C. having a basis weight of 120 gsm (4.08 osy).

Specimen 4 was a layer of BASF E123-199 bipolar superabsorbent material available from BASF Corporation of Ludwigshafen, Germany having a basis weight of 120 gsm (4.08 osy).

Specimen 5 was a 120 gsm (4.08 osy) layer of OASIS 101 superabsorbent fibers available from Technical Absorbents of Great Coates, Grimsby, United Kingdom.

Specimen 6 was a 2 mm (0.08 inch) thick layer of RYNEL type 562-B medical grade hydrophilic polyurethane foam available from Rynel Inc. of Booth Bay, Me.

Specimen 7 was a 3 mm (0.12 inch) thick layer of RYNEL type 562-B medical grade hydrophilic polyurethane foam available from Rynel Inc. of Booth Bay, Me., with 50% by weight HYSORB 8800 superabsorbent material available from BASF Corporation of Charlotte, N.C.

Specimen 8 was a composite layer of material comprising a top layer of 2.45 osy (83.3 gsm) TABCW surge material, a middle layer of bipolar superabsorbent material identical to Specimen 4 described above, and a bottom layer of 2.45 osy (83.3 gsm) TABCW surge material. The size of specimen was 4 inches (10.2 cm) by 14 inches (35.6 cm) with an evaporative surface area of 56 square inches (361 square cm). The smaller samples used in conducting the Evaporation Rate Test were cut from the larger specimen such that the top and bottom layers of each sample did not extend around the side edges of the middle layer.

The evaporation rate for each specimen was determined using the Evaporation Rate Test set forth previously. The results were recorded and are provided in Table 1. The control specimen representing conventional absorbent body constructions that include cellulosic fibers (e.g., wood pulp fluff), Specimen 1, had an evaporative rate of 0.221 g/hr/in². The remaining specimens, constructed without cellulosic fibers, each had an evaporation rate substantially greater than the control specimen. While the specimen constructed of TABCW surge material (Specimen 2) yielded the highest evaporation rate, the surge material cannot retain liquid under pressure.

TABLE 1

| | | | | Amount | |
|---|---|---|---|---|---|
| | | | Evaporation | Evaporated | Evaporation |
| Absorbent | | | Rate | in Two Hours | % of Initial |
| Specimen | Weight | | (g/hr/in²) | (grams) | Fluid Amount |
| Specimen 1 | 18.04 | osy | 0.221 | 25 | 28% |
| Specimen 2 | 3.5 | osy | 0.794 | 89 | 99% |
| Specimen 3 | 0.31 | g | 0.440 | 49 | 55% |
| Specimen 4 | 0.31 | g | 0.458 | 51 | 57% |
| Specimen 5 | 0.306 | g | 0.481 | 54 | 60% |
| Specimen 6 | 8 | osy | 0.736 | 82 | 92% |
| Specimen 7 | 27 | osy | 0.491 | 55 | 61% |
| Specimen 8 | 0.702 | osy | 0.398 | 45 | 50% |

### Experiment 2

A second test was conducted to determine the effect that various outer cover materials have on evaporation rate. For each outer cover material to be tested, the Evaporation Rate Test was conducted using the TABCW surge material set forth in Specimen 2 of Experiment 1. The specimen of the outer cover being tested was sized to completely cover the absorbent sample and extend beyond the edge of the absorbent sample by at least one inch (2.54 cm) on each side of the absorbent sample. The particular outer cover specimens tested were all 6 inches (15.2 cm) by 6 inches (15.2 cm). The specimen was placed over (in direct contact therewith) a saturated 2 inch (5.1 cm) by 2 inch (5.1 cm) sample of 3.5 osy (119 gsm) surge layer material, identical to the material of Sample 2 in Experiment 1, on the hot plate for the duration of the Evaporation Rate Test. The various materials tested included the following:

Specimen 1 was a microporous film/nonwoven laminate comprising a spunbond nonwoven material layer laminated to a microporous film having a WVTR of 1500 g/m²/24. The laminate comprised a 15.5 gsm (0.45 osy) 2 dpf polypropylene spunbond adhesively laminated to a moderately breathable 17 gsm (0.5 osy) polyethylene-based film to yield a WVTR if 1500 g/m²/24. As is known in the art, the weight percentage of calcium carbonate loading and stretching conditions are adjusted to achieve the desired level of WVTR

Specimen 2 was a laminate similar to Specimen 1 but having the weight percentage of calcium carbonate and the stretching conditions adjusted to achieve a WVTR of 6000 g/m²/24.

Specimen 3 was a laminate similar to Specimen 1 but having the weight percentage of calcium carbonate and the stretching conditions adjusted to achieve a WVTR of 12,000 g/m²/24.

Specimen 4 was a laminate similar to Specimen 1 but having the weight percentage of calcium carbonate and the stretching conditions adjusted to achieve a WVTR of 15,000 g/m²/24.

Specimen 5 was a polyurethane film treated with adhesive to be used as a highly breathable wound dressing manufactured by 3M Company of St. Paul, Minn., U.S.A.

Specimen 6 was 1996 Guilford Loop Tricot Polyester material with raised loop construction available from Guilford Mills Incorporated of Greensboro, N.C. .

Specimen 7 was 0.65 osy (22.1 gsm) spunbond meltblown spunbond (SMS) material used as containment flaps in HUGGIES diapers available from Kimberly-Clark Worldwide, Inc. of Neenah, Wis.

Specimen 8 was a 0.5 osy (17 gsm) polypropylene spunbond body-side liner used as the bodyside liner in HUGGIES diapers manufactured by Kimberly-Clark Worldwide, Inc. of Neenah, Wis.

Specimen 9 was a polypropylene spunbond material with 5 weight percent granular ceramic material added to one side of the spunbond. The material was manufactured by ThermoFlow Health Products Ltd of Vancouver, British Columbia. The specimen was tested with the ceramic material side facing up and exposed to air and the non-ceramic material side facing down and adjacent the saturated test sample.

Specimen 10 was the same material of Specimen 10 but with the ceramic material side facing down and adjacent the saturated test sample and the non-ceramic material side facing up and exposed to air.

The evaporation rate for each specimen was determined using the Evaporation Rate Test set forth previously. The top surface of the specimen exposed to air was examined after the measurement for the Evaporation Rate Test were completed to determine if the specimen felt wet or damp to the touch. The results were recorded and are provided in Table 2. In general, as the WVTR of the outer cover material increased, the evaporation rate of the surge material was less effected. That is, the evaporative capacity of the surge material increased. However, the increase in evaporative capacity of the surge material was substantially less once the WVTR of the outer cover material exceeded 12,000.

TABLE 2

| | | | | Amount | |
|---|---|---|---|---|---|
| | | | Evaporation | Evaporated in | Evaporation |
| Outer Cover | Basis | | Rate | Two Hours | % of Initial |
| Specimen | Weight | | (g/hr/in²) | (grams) | Fluid Amount |
| Uncovered | N/A | | 0.794 | 89 | 99% |
| Specimen 1 | N/A | | 0.239 | 27 | 30% |
| Specimen 2 | N/A | | 0.349 | 39 | 43% |
| Specimen 3 | N/A | | 0.4725 | 53 | 59% |
| Specimen 4 | N/A | | 0.489 | 55 | 61% |
| Specimen 5 | 7.86 | osy | 0.262 | 29 | 33% |
| Specimen 6 | 1.625 | osy | 0.684 | 77 | 85% |
| Specimen 7 | 0.65 | osy | 0.606 | 68 | 75% |
| Specimen 8 | 0.5 | osy | 0.754 | 84 | 94% |
| Specimen 9 | 0.7 | osy | 0.61 | 68 | 76% |
| Specimen 10 | 0.7 | osy | 0.488 | 55 | 61% |

### Experiment 3

A third test was conducted to determine the evaporation rates of various specimens comprises of a combination of an absorbent body specimen and an outer cover material specimen.

A total of six combinations were tested.

Specimen 1 was a control sample comprising the absorbent body of Specimen 1 of Experiment 1 and the outer cover material of Specimen 1 of Experiment 2. This specimen represents the absorbent body and outer cover construction used in HUGGIES SUPREME diapers currently available from Kimberly-Clark Worldwide, Inc. of Neenah, Wis.

Specimen 2 comprised the absorbent body of Specimen 6 of Experiment 1 and the outer cover material of Specimen 3 of Experiment 2.

Specimen 3 comprised the absorbent body of Specimen 7 of Experiment 1 and the outer cover material of Specimen 3 of Experiment 2.

Specimen 4 comprised the absorbent body of Specimen 9 of Experiment 1 and the outer cover material of Specimen 3 of Experiment 2.

Specimen 5 comprised the absorbent body of Specimen 2 of Experiment 1 and the outer cover material of Specimen 4 of Experiment 2.

The Evaporation Rate Test was conducted for each Specimen, with the outer cover for each specimen having a size of 6 inches (15.2 cm) by 6 inches (15.2 cm) and overlaying the absorbent body in direct contact therewith. The results of Experiment 3 are set forth in Table 3. The control sample, Specimen 1, had an evaporation rate of 0.128 g/hr/in². The remaining specimens each had an evaporation rate exceeding 0.15 g/hr/in².

TABLE 3

| Outer Cover/ | | Amount Evaporated | Evaporation |
|---|---|---|---|
| Absorbent Body | Evaporation | in Two Hours | % of Initial |
| Specimen | Rate (g/hr/in²) | (grams) | Fluid Amount |
| Specimen 1 | 0.128 | 14 | 16% |
| Specimen 2 | 0.434 | 49 | 54% |
| Specimen 3 | 0.389 | 44 | 48% |
| Specimen 4 | 0.320 | 36 | 40% |
| Specimen 5 | 0.489 | 55 | 61% |

When introducing elements of the present invention or the preferred aspect(s) thereof, the articles “a”, “an”, “the”, and “said” are intended to mean that there are one or more of the elements. The terms “comprising,” “including”, and “having” are intended to be inclusive and mean that there may be additional elements other than the listed elements.

As various changes could be made in the above constructions without departing from the scope of the invention, it is intended that all matter contained in the above description or illustrated in the accompanying drawings shall be interpreted as illustrative and not in a limiting sense.

What is claimed is:

1. A disposable garment for personal wear, said garment comprising:
a liquid permeable liner having a bodyfacing surface for facing a wearer of the garment;
a liquid impermeable and vapor permeable outer cover in opposed relationship with the liner, said outer cover having a water vapor transmission rate (WVTR) as determined by a Water Vapor Transmission Rate Test of at least about 6,000 g/m²/24 hours; and
an absorbent body disposed between the liner and the outer cover, the disposable garment having an evaporation rate as determined by an Evaporation Rate Test of at least 0.15 g/hr/in².

2. The disposable garment set forth in claim 1 wherein the disposable garment has an evaporation rate as determined by the Evaporation Rate Test of at least about 0.30 g/hr/in².

3. The disposable garment set forth in claim 2 wherein the disposable garment has an evaporation rate as determined by the Evaporation Rate Test of at least about 0.40 g/hr/in².

4. The disposable garment set forth in claim 1 wherein the outer cover has a water vapor transmission rate as determined by a Water Vapor Transmission Rate Test of at least about 12,000 g/m²/24.

5. The disposable garment set forth in claim 4 wherein the outer cover has a water vapor transmission rate as determined by a Water Vapor Transmission Rate Test of at least about 15,000 g/m²/24 hours.

6. The disposable garment set forth in claim 1 wherein the absorbent body comprises a retention layer free from cellulosic fibers.

7. The disposable garment set forth in claim 6 wherein the retention layer comprises superabsorbent material.

8. The disposable garment set forth in claim 6 wherein the retention layer comprises a foam.

9. The disposable garment set forth in claim 6 wherein the absorbent body further comprises an outer separation layer disposed between the retention layer and the outer cover, said outer separation layer facilitating distribution of liquid over the outer cover within the garment.

10. The disposable garment set forth in claim 9 wherein the absorbent body further comprises an inner separation layer disposed between the retention layer and the liner.

11. The disposable garment set forth in claim 9 wherein the outer separation layer comprises a surge material.

12. The disposable garment set forth in claim 11 wherein the surge material comprises a through-air bonded carded web.

13. The disposable garment set forth in claim 6 wherein the absorbent body further comprises a wrapsheet surrounding the retention layer, said wrapsheet comprising a surge material.

14. The disposable garment set forth in claim 1 wherein the absorbent body has a saturation capacity ratio as determined by a Saturation Capacity Test of at least about 17 grams/gram.

15. The disposable garment set forth in claim 14 wherein the absorbent body has a saturation capacity ratio as determined by the Saturation Capacity Test in the range of about 17 grams/gram to about 30 grams/gram.

16. A disposable garment for personal wear, said garment comprising:
a liquid permeable liner having a bodyfacing surface for facing a wearer of the garment;
a liquid impermeable and vapor permeable outer cover in opposed relationship with the liner, said outer cover having a water vapor transmission rate (WVTR) as determined by a Water Vapor Transmission Rate Test of at least about 6,000 g/m²/24 hours; and
an absorbent body disposed between the liner and the outer cover, the absorbent body having an evaporation rate as determined by an Evaporation Rate Test of at least 0.30 g/hr/in².

17. The disposable garment set forth in claim 16 wherein the absorbent body has an evaporation rate as determined by an Evaporation Rate Test of at least 0.35 g/hr/in².

18. The disposable garment set forth in claim 17 wherein the absorbent body has an evaporation rate as determined by an Evaporation Rate Test of at least about 0.40 g/hr/in².

19. The disposable garment set forth in claim 16 wherein the outer cover has a water vapor transmission rate as determined by a Water Vapor Transmission Rate Test of at least about 12,000 g/m²/24.

20. The disposable garment set forth in claim 16 wherein the outer cover has a water vapor transmission rate as determined by a Water Vapor Transmission Rate Test of at least about 15,000 g/m²/24 hours.

21. The disposable garment set forth in claim 16 wherein the absorbent body comprises a retention layer free from cellulosic fibers.

22. The disposable garment set forth in claim 16 wherein the absorbent body has a saturation capacity ratio as determined by a Saturation Capacity Test of at least about 17 grams/gram.

23. The disposable garment set forth in claim 22 wherein the absorbent body has a saturation capacity ratio as determined by the Saturation Capacity Test in the range of about 17 grams/gram to about 30 grams/gram.

24. A disposable garment for personal wear, said garment comprising:
a liquid permeable liner having a bodyfacing surface for facing a wearer of the garment;
a liquid impermeable and vapor permeable outer cover in opposed relationship with the liner; and
an absorbent body disposed between the liner and the outer cover, the absorbent body having an evaporation rate as determined by an Evaporation Rate Test of at least 0.30 g/hr/in²., and a saturation capacity ratio as determined by a Saturation Capacity Test of at least about 17 grams/gram,
the disposable garment having an evaporation rate as determined by the Evaporation Rate Test of at least 0.15 g/hr/in².

25. The disposable garment set forth in claim 24 wherein the disposable garment has an evaporation rate as determined by the Evaporation Rate Test of at least about 0.30 g/hr/in².

26. The disposable garment set forth in claim 24 wherein the disposable garment has an evaporation rate as determined by the Evaporation Rate Test of at least about 0.40 g/hr/in².

27. The disposable garment set forth in claim 24 wherein the absorbent body has a saturation capacity ratio as determined by the Saturation Capacity Test in the range of about 17 grams/gram to about 30 grams/gram.

28. A disposable garment for personal wear, said garment comprising:
a liquid permeable liner having a bodyfacing surface for facing a wearer of the garment;
a liquid impermeable and vapor permeable outer cover in opposed relationship with the liner; and
an absorbent body disposed between the liner and the outer cover, the absorbent body having an evaporation rate as determined by an Evaporation Rate Test of at least 0.30 g/hr/in²., and a saturation capacity as determined by a Saturation Capacity Test of at least about 50 grams,
the disposable garment having an evaporation rate as determined by the Evaporation Rate Test of at least 0.15 g/hr/in².

29. The disposable garment set forth in claim 28 wherein the absorbent body has a saturation capacity as determined by the Saturation Capacity Test in the range of about 50 to about 500 grams.

30. The disposable garment set forth in claim 28 wherein the absorbent body has a saturation capacity as determined by the Saturation Capacity Test in the range of about 200 to about 500 grams.

31. The disposable garment set forth in claim 28 wherein the absorbent body has a saturation capacity as determined by the Saturation Capacity Test in the range of about 50 to about 350 grams.

32. The disposable garment set forth in claim 28 wherein the absorbent body has a saturation capacity as determined by the Saturation Capacity Test in the range of about 50 to about 250 grams.

33. The disposable garment set forth in claim 28 wherein the disposable garment has an evaporation rate as determined by the Evaporation Rate Test of at least about 0.30 g/hr/in².
